(19) United States
(12) Patent Application Publication
(10) Pub. No.: US 2007/0282142 A1
(43) Pub. Date: Dec. 6, 2007

(54) Gas Production Facility, Gas Supply Container, And Gas For Manufacture Of Electronic Devices

(75) Inventors: Tadahiro Ohmi, Sendai-shi (JP); Yasuyuki Shirai, Sendai-shi (JP); Takeyoshi Kato, Tokyo (JP); Kimiaki Tanaka, Tokyo (JP); Masahiro Nakamura, Tokyo (JP); Katsutomo Tanaka, Tokyo (JP)

Correspondence Address: KRATZ, QUINTOS & HANSON, LLP, 1420 K Street, N.W., Suite 400, WASHINGTON, DC, 20005, US

(73) Assignee: ZEON CORPORATION, Sendi-shi (JP)

(21) Appl. No.: 10/592,278

(22) PCT Filed: Feb. 16, 2005
(86) PCT No.: PCT/JP05/02329

§ 371 Date: May 22, 2007

(30) Foreign Application Priority Data
Mar. 10, 2004 (JP) 2004-068018

(51) Int. Cl. C07C 19/08; A61L 9/00

(52) U.S. Cl. 570/124; 422/305

(57) ABSTRACT

An apparatus for producing a gas using a raw material gas having high reactivity, in particular, a fluorinated hydrocarbon, or a vessel for supplying the gas, characterized in that the surface of a portion thereof contacting with the gas has an average roughness of 1 μm or less in terms of a center line average roughness Ra. It is preferred that an oxide-based passivated film such as a film based on chromium oxide, aluminum oxide, yttrium oxide, magnesium oxide or the like is formed on the surface having a roughness controlled as above. The above apparatus and vessel can be suitably used for preventing the contamination of a raw material gas originated from a gas production apparatus or a vessel for supplying the gas.

### TECHNICAL FIELD

This invention relates to a gas production facility, a gas supply container, and an electronic device manufacturing gas that are useful in the field of manufacturing electronic devices. More specifically, this invention relates to a facility from a final production process to filling into a container of a gas (also including a liquefied gas) for use in carrying out the processing that uses a plasma, a supply container and a gas for plasma reaction.

### BACKGROUND ART

In recent years, following the increase in level and performance of electronic devices, high-purification production techniques for raw materials to be used have been getting important. Particularly, in the manufacture of semiconductor devices, the ppb (parts per billion) level impurity management has been required for raw materials to be used.

However, there has been a problem that the current impurity management of the raw materials for the manufacture of semiconductor devices cannot be said to be sufficient.

In a semiconductor manufacturing apparatus such as a plasma CVD apparatus and a facility attendant thereon, the impurities, as described above, are generated on the inner surfaces of facilities, pipes, and components that contact with a gas used in the manufacture, due to decomposition and reaction of the gas caused by catalysis reaction with the inner surfaces or the incorporation of moisture and gas components caused by shortage of cleaning of the inner surfaces.

Techniques for preventing the generation of such impurities are proposed, for example, in Japanese Unexamined Patent Application Publication (JP-A) No. H7-233476 (U.S. Pat. No. 5,951,787) (Patent Document 1), Japanese Unexamined Patent Application Publication (JP-A) No. H11-302824 (Patent Document 2), and so on. Among them, Patent Document 1 discloses a passivation film forming method of coating a passivation film in the form of a chromium oxide film on the surface of a gas contact portion formed of ferritic stainless steel, in order to prevent generation of corrosion products caused by contact with a halogen-based corrosive gas.

On the other hand, Patent Document 2 discloses a fluid supply system such as a pipe that is formed with a passivation film made of aluminum oxide on its aluminum-containing stainless steel surface, thereby safely supplying a highly corrosive fluid.

Patent Document 1:
Japanese Unexamined Patent Application Publication (JP-A) No. H7-233476

Patent Document 2:
Japanese Unexamined Patent Application Publication (JP-A) No. H 11-302824

### DISCLOSURE OF THE INVENTION

### Problem to be Solved by the Invention

As described above, Patent Documents 1 and 2 disclose the formation of the passivation film on the surface of the using apparatus that uses a gas, such as a pipe for supplying a gas or a process apparatus that carries out the processing using a gas. However, actually, assuming that the impurities are incorporated at a time point when a feed gas is produced or when a feed gas is placed in a supply container, even if the generation of the impurities is suppressed on the using apparatus side like in Patent Documents 1 and 2, it is not possible to prevent a bad influence caused by the impurities. That is, in Patent Documents 1 and 2, there is no discussion about the bad influence due to the contamination in the feed gas.

Further, in Patent Documents 1 and 2, there is also no discussion at all about contamination of a gas contact surface due to a specific highly reactive feed gas, for example, a fluorinated carbon compound, or a specific relationship between surface roughness and impurities on the surface that contacts the feed gas.

An object of this invention is to provide an electronic device manufacturing gas production facility and a supply container that can reduce incorporation of impurities such as moisture in the state of a feed gas and decomposition/dissociation of the feed gas and thus is sufficiently effective for achieving higher performance/higher reliability of a semiconductor device, an electronic device manufacturing gas production method, and an electronic device manufacturing gas.

Still another object of this invention is to provide an electronic device feed gas production apparatus that can reduce contamination when producing a fluorinated carbon compound as a feed gas.

### Means for Solving the Problem

As a result of conducting diligent studies in order to accomplish the foregoing objects, the present inventors have found that the roughness and material of the inner surfaces of a feed gas production facility and supply facility largely affect the impurity content of a feed gas and setting them in proper ranges is effective for realizing high purification of a fluorinated carbon compound for use in carrying out the processing that uses a plasma, and have reached the completion of this invention.

For example, in the case of manufacturing a semiconductor device, if impurities such as moisture are contained in a gas when heat treatment is applied to a semiconductor element having an interlayer insulating film obtained by plasma CVD (Chemical Vapor Deposition) or the like, a corrosive gas is generated and adversely affects the reliability of the semiconductor device.

Thus, according to this invention, there are obtained a gas production facility and a gas supply container, wherein a surface roughness of a portion of each of the gas production facility and the gas supply container that contacts with a gas for manufacturing an electronic device is 1 μm or less in terms of a center line average roughness Ra.

Further, according to this invention, there are provided a gas production facility and a gas supply container, wherein an oxide passivation film is formed on the inner surface of the electronic device manufacturing gas production facility.

The oxide passivation film of the production facility is preferably chromium oxide, aluminum oxide, titanium oxide, yttrium oxide, or magnesium oxide.

Further, according to this invention, there are provided a gas production facility and a gas supply container, wherein the gas for manufacturing the electronic device comprises a fluorinated carbon compound of which a ratio (F/C ratio) between the number of fluorine atoms and the number of carbon atoms is 1.0 to 2.0.

Further, there are obtained a method of producing a fluorinated carbon compound and a method of supplying a fluorinated carbon compound, wherein the foregoing gas production facility and gas supply container are used, respectively.

Further, there is provided a gas for manufacturing an electronic device, which is produced by the use of the foregoing gas production facility and having a moisture content of 50 vol ppb or less.

### Effect of the Invention

According to this invention, there are obtained a production method and a supply method each being sufficiently effective for high purification of an electronic device manufacturing feed gas, particularly a fluorinated carbon compound.

Further, a film on a substrate formed by CVD using an electronic device manufacturing gas of this invention is hardly subjected to film stripping or metal corrosion due to generation of hydrogen fluoride.

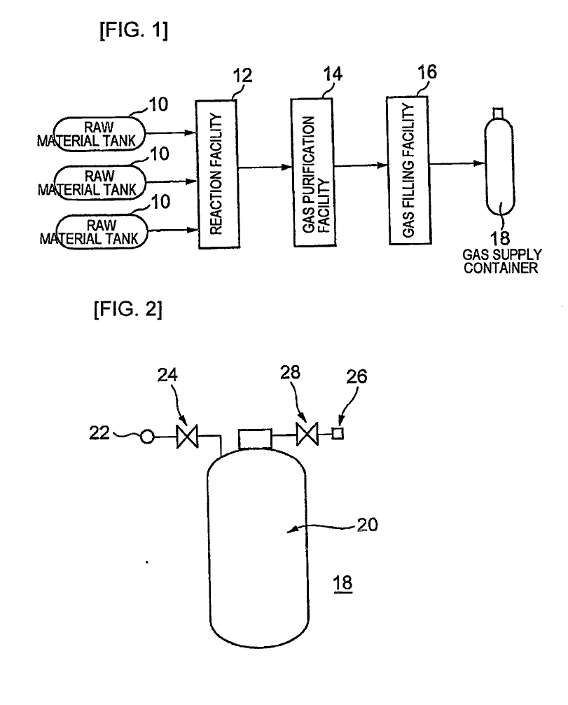

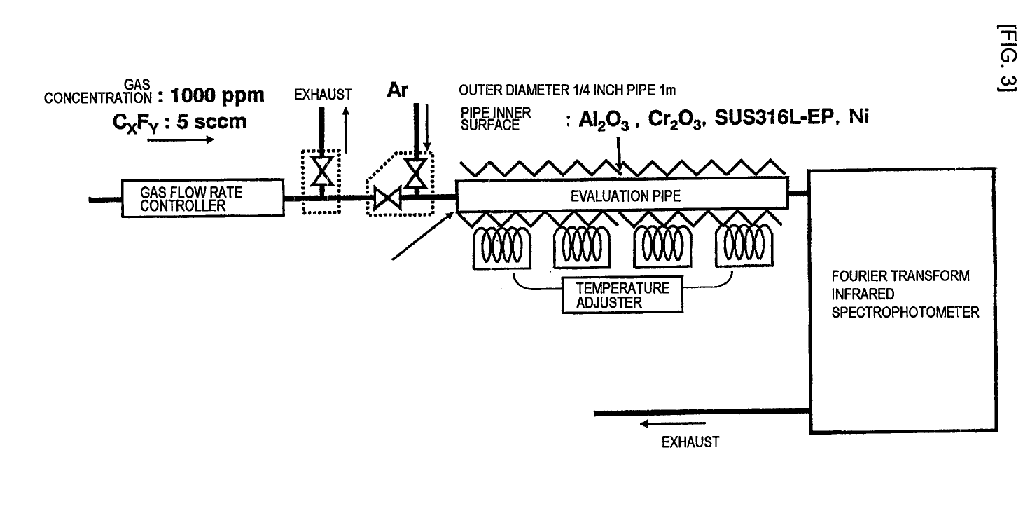

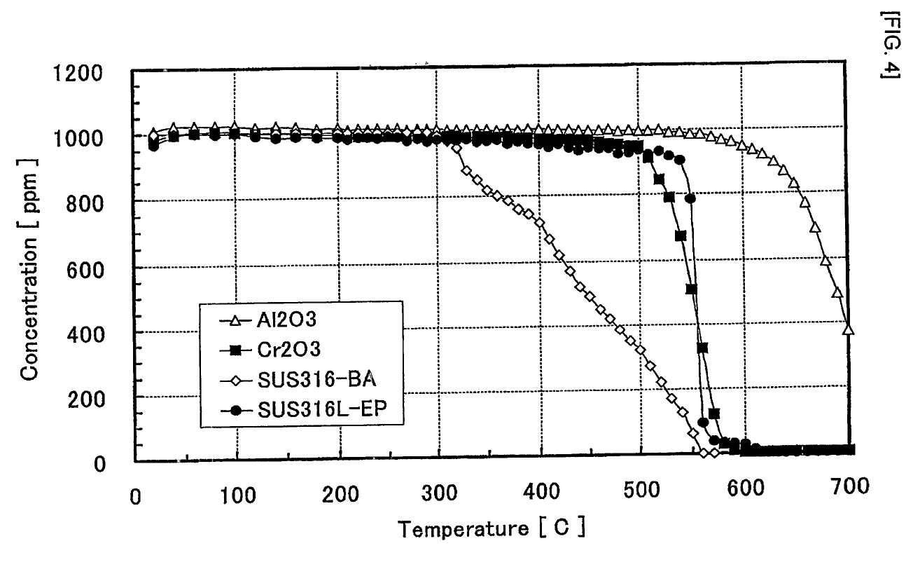

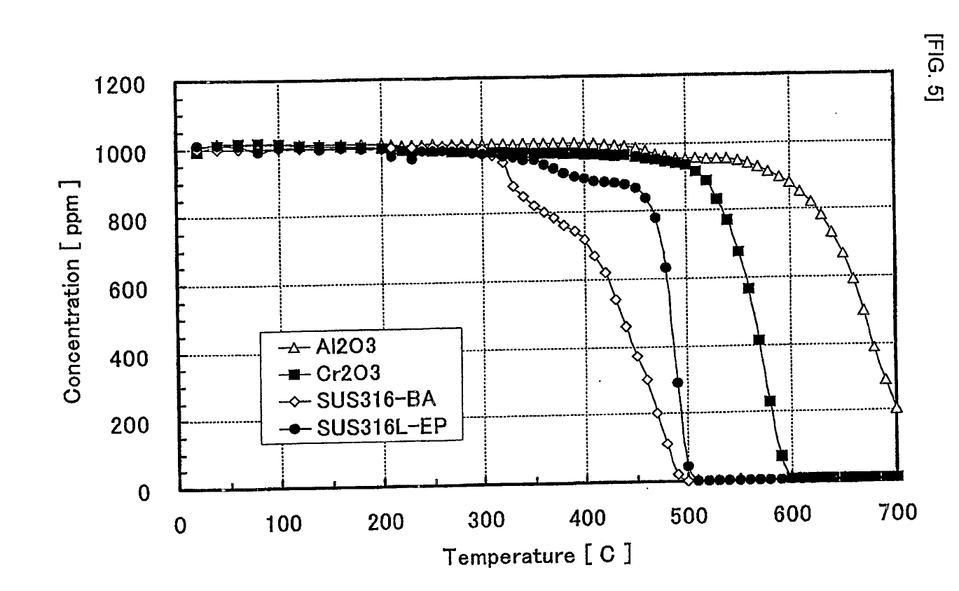

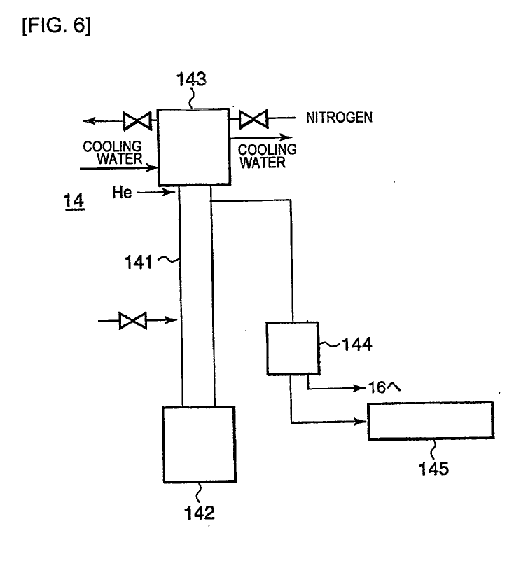

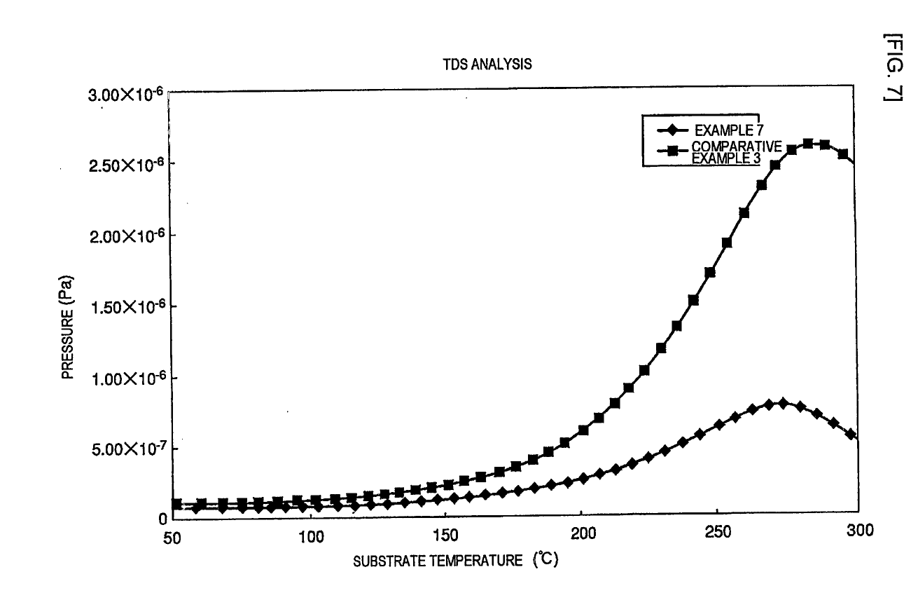

### BRIEF DESCRIPTION OF THE DRAWINGS

[FIG. 1] is a block diagram showing one example of a gas production facility applicable with this invention.

[FIG. 2] is a diagram showing the structure of a gas supply container shown in FIG. 1.

[FIG. 3] is a diagram for explaining an evaluation apparatus adapted to evaluate thermal decomposition characteristics of a fluorinated carbon compound of a passivation film according to this invention.

[FIG. 4] is a diagram showing the evaluation results in the case where octafluorocyclopentene was used as a fluorinated carbon compound with respect to the evaluation apparatus shown in FIG. 3.

[FIG. 5] is a diagram showing the evaluation results in the case where octafluoro-2-pentyne was used as a fluorinated carbon compound with respect to the evaluation apparatus shown in FIG. 3.

[FIG. 6] is a diagram showing a gas purification facility of the gas production facility shown in FIG. 1.

[FIG. 7] is a diagram showing the results of thermal desorption spectroscopy (TDS analysis) of a film obtained on a substrate in Example 7 and a film obtained on a substrate in Comparative Example 3.

### DESCRIPTION OF SYMBOLS

10 raw material tank

12 reaction facility

14 gas purification facility

16 gas filling facility

18 gas supply container

### BEST MODE FOR CARRYING OUT THE INVENTION

Referring to FIG. 1, description will be made of one example of a gas production facility applicable with this invention. As illustrated, the gas production facility comprises a plurality of raw material tanks 10, a reaction facility 12, a gas purification facility 14, and a gas filling facility 16. In this gas production facility, raw materials from the plurality of raw material tanks 10 are reacted in the reaction facility 12 and then purified in the gas purification facility 14, and a purified feed gas is filled into a gas supply container 18 by the gas filling facility 16. Herein, the gas supply container 18 comprises, as shown in FIG. 2, a container body 20, a joint 22 for connection to the gas filling facility 16, a valve 24 provided between the joint 22 and the container body 20, a joint 26 for connection to an electronic device manufacturing facility (not shown), and a valve 28 provided between the joint 26 and the container body 20.

The effect can be achieved by applying this invention to at least the gas purification facility 14 and the gas filling facility 16 in the gas production facility and, further, the effect can be achieved by applying this invention to a gas contact surface of the gas supply container 18. As a material of the gas production facility and the gas supply container 18 described above, a stainless steel or an aluminum alloy is applied. Particularly, as the stainless steel, use can be made of an austenitic, ferritic, austenitic-ferritic, or martensitic stainless steel and, for example, use is preferably made of austenitic SUS304, SUS304L, SUS316, SUS316L, SUS317, SUS317L, or the like. As surface polishing of the stainless steel, it is possible to carry out pickling, mechanical polishing, belt polishing, barreling, buffing, fluidized abrasive polishing, lapping, burnishing, chemical polishing, electrochemical polishing, electrolytic polishing, or the like, which may of course be used in combination thereof for the single stainless steel.

In this case, buffing, fluidized abrasive polishing, lapping, burnishing, chemical polishing, electrochemical polishing, or electrolytic polishing is effective wherein the center line average roughness Ra (Ra is defined in Japanese Industrial Standard JIS B0601 and also disclosed in United States Patent No. U.S. Pat. No. 6,544,893 B2) of the surface of a portion that contacts an electronic device manufacturing gas is 1 μm or less. Although the foregoing center line average roughness Ra is 1 μm or less, it is preferably 0.7 μm or less and particularly preferably 0.5 μm or less. When the center line average roughness Ra is greater than the foregoing range, there is a possibility that impurity gases, particles, and so on adsorbed on the inner wall of the container are incorporated into the electronic device manufacturing gas.

It is preferable that an oxide passivation film be formed on the inner surfaces of portions, which contact the electronic device manufacturing gas, of the gas production facility and the supply container in this invention. This is because if it is not formed, even the stainless steel applied with the surface cleaning treatment such as electrolytic polishing causes decomposition or dissociation of a highly reactive gas due to catalysis on the metal surface. It is more preferable that there be formed, among oxide passivation films, an oxide passivation film of at least one selected from the group consisting of aluminum oxide, chromium oxide, titanium oxide, yttrium oxide, and magnesium oxide, and it is particularly preferable that an oxide passivation film made of aluminum oxide be formed in terms of corrosion resistance of the material and reduction in moisture adsorption amount on the inner surface. By forming the oxide passivation film on the inner surface of the portion that contacts the electronic device manufacturing gas, it is possible to improve the corrosion resistance and to reduce the moisture adsorption amount on the surface. The oxide passivation film can be formed by contacting an oxidizing gas with the portion, which contacts the electronic device manufacturing gas, of each of the gas production facility and the supply container and applying heat treatment thereto.

For example, in the case of an oxide passivation film made of aluminum oxide, by contacting an oxidizing gas with the surface of an aluminum-containing stainless steel and carrying out heat treatment, it is possible to form a passivation film made of aluminum oxide which does not contain any other metal oxide. By forming the aluminum oxide passivation film excellent in corrosion resistance on the surface of the aluminum-containing stainless steel, it is possible to overcome the conventional problem of workability and hardness and to form the aluminum oxide passivation film suitable for the pipe material or the like for use in the gas supply container and the gas production facility.

An oxide passivation film is formed by contacting an aluminum-containing stainless steel or the like with an oxidizing gas containing oxygen or moisture. When forming a passivation film made of aluminum oxide which does not contain any other metal oxide, the oxygen concentration in the oxidizing gas is preferably 500 vol ppb to 100 vol ppm and particularly preferably 1 vol ppm to 50 vol ppm, or the moisture concentration is preferably 200 vol ppb to 50 vol ppm and particularly preferably 500 vol ppb to 10 vol ppm. Further, a mixed gas containing hydrogen may be used in the oxidizing gas. The aluminum-containing stainless steel contains, in addition to aluminum, stainless steel components such as iron, chromium, and nickel. Accordingly, if the oxidizing component is present in large amount, the other metals are also oxidized along with aluminum and hence it is difficult to form the aluminum oxide passivation film containing no other metal oxide. On the other hand, if the oxidizing component is too small in amount, the oxide film cannot be formed.

Further, the oxidation treatment temperature is 700° C. to 1200° C. and preferably 800° C. to 1100° C. When forming the aluminum oxide passivation film containing no other metal oxide, by carrying out the oxidation at the foregoing temperature, it is possible to prevent oxidation of the other metals and to selectively oxidize only aluminum. If the oxidation treatment temperature is below the foregoing range, iron and chromium are also oxidized, while, if it is above the foregoing range, crystals of aluminum oxide are deposited on the surface of the formed aluminum oxide passivation film and, when a fluid is supplied, the deposited aluminum oxide crystals are stripped or cracked and hence there is a possibility of contamination of the supplied fluid.

Even in a more excessive oxidizing atmosphere, by adding reducing hydrogen to the oxidizing gas, it becomes possible to widely set the concentration of the oxidizing component in the oxidizing atmosphere. By adding hydrogen to the oxidizing gas, a finer and stronger aluminum oxide passivation film can be formed.

According to the foregoing oxide passivation film forming method, the oxidation treatment time is normally only 30 minutes to 3 hours and no labor is required for applying heat treatment after aluminum coating, which, however, was conventionally required, so that the productivity can be improved.

Further, the oxide passivation film of this invention may be a thermally sprayed film (a film formed on the surface by thermally spraying a passivation oxide). The thermally sprayed film is formed by cleaning the inner surface of the portion that contacts the electronic device manufacturing gas and then spraying the passivation oxide in a molten state onto the inner surface (thermal spraying process). As a thermal spraying method, use can be made of a conventionally known method such as plasma spraying or arc spraying. When forming the thermally sprayed oxide passivation film on the inner surface of the portion that contacts the electronic device manufacturing gas, a thermally sprayed metal film may be formed as an undercoat of the thermally sprayed oxide passivation film in order to improve the adhesion.

In this invention, when welding a pipe applied with the aluminum oxide passivation film, it is preferable to add an oxidizing gas containing oxygen or moisture to a back shield gas so as to form an aluminum oxide passivation film on the surface of a welding portion simultaneously with the welding. In the back shield gas, the oxygen concentration is preferably 10 vol ppm to 5000 vol ppm or the moisture concentration is preferably 1 vol ppm to 1000 vol ppm. Further, the oxidizing gas may be an oxidizing mixed gas containing hydrogen.

In the manner as described above, it is possible to prevent local degradation in the vicinity of the welding portion, which cannot be overcome conventionally, and further, since the aluminum oxidation/passivation process is enabled simultaneously with the welding without carrying out such a process again after the welding, it is possible to improve the productivity.

As a result, the aluminum oxide passivation film more excellent in corrosion resistance than the chromium oxide passivation film can be formed in a short time and at a low cost so that it becomes possible to construct a fluid supply system that can stably supply a highly corrosive fluid.

The electronic device manufacturing gas applied to this invention is not limited, but this invention is particularly effective for an electronic device manufacturing gas composed of a fluorinated carbon compound. The fluorinated carbon compound represents a compound composed of only carbon atoms and fluorine atoms. The fluorinated carbon compound is preferably a compound having a double bond or a triple bond.

It is known that the fluorinated carbon compound is used in forming an insulating film or an interlayer insulating film by plasma dry etching or plasma CVD in the electronic device manufacturing process. Particularly for the formation of the insulating film or the interlayer insulating film, use is preferably made of a fluorinated carbon compound of which the ratio (hereinafter abbreviated as the F/C ratio) between the number of fluorine atoms and the number of carbon atoms is 1.0 to 2.0 and preferably 1.2 to 1.8. If the F/C ratio is smaller than this range, the insulating properties of the formed film are degraded, while, when it exceeds this range, the film forming rate is degraded.

The carbon number of the fluorinated carbon compound is preferably 2 to 7, more preferably 2 to 6, further preferably 2 to 5, and particularly preferably 4 to 5. As specific examples of the fluorinated carbon compound, there are cited a fluorinated carbon compound having a carbon number of 2, such as tetrafluoroethylene, a fluorinated carbon compound having a carbon number of 3, such as hexafluoropropene, tetrafluoropropyne, or tetrafluorocyclopropene, a fluorinated carbon compound having a carbon number of 4, such as hexafluoro-2-butyne, hexafluoro-1-butyne, hexafluorocyclobutene, hexafluoro-1,3-butadiene, hexafluoro-(1-methylcyclopropene), octafluoro-1-butene, or octafluoro-2-butene, a fluorinated carbon compound having a carbon number of 5, such as octafluoro-1-pentyne, octafluoro-2-pentyne, octafluoro-1,3-pentadiene, octafluoro-1,4-pentadiene, octafluorocyclopentene, octafluoroisoprene, hexafluorovinylacetylene, octafluoro-(1-methylcyclobutene), or octafluoro-(1,2-dimethylcyclopropene), a fluorinated carbon compound having a carbon number of 6, such as dodecafluoro-1-hexene, dodecafluoro-2-hexene, dodecafluoro-3-hexene, decafluoro-1,3-hexadiene, decafluoro-1,4-hexadiene, decafluoro-1,5-hexadiene, decafluoro-2,4-hexadiene, decafluorocyclohexene, hexafluorobenzene, octafluoro-2-hexyne, octafluoro-3-hexyne, octafluorocyclo-1,3-hexadiene, or octafluorocyclo-1,4-hexadiene, and a fluorinated carbon compound having a carbon number of 7, such as undecafluoro-1-heptene, undecafluoro-2-heptene, undecafluoro-3-heptene, or dodecafluorocycloheptene.

Among these fluorinated carbon compounds, tetrafluoroethylene, hexafluoropropene, tetrafluoropropyne, hexafluorocyclobutene, hexafluoro-1,3-butadiene, hexafluoro-1-butyne, hexafluoro-2-butyne, octafluorocyclobutane, octafluorocyclopentene, octafluoro-1,3-pentadiene, octafluoro-1,4-pentadiene, octafluoro-1-pentyne, octafluoro-2-pentyne, and hexafluorobenzene are preferable, octafluorocyclopentene, octafluoro-2-pentyne, octafluoro-1,4-pentadiene, and hexafluoro-1,3-butadiene are more preferable, and octafluoro-2-pentyne and octafluorocyclopentene are particularly preferable.

In this invention, by the use of a rectifier with particularly high airtightness (hereinafter referred to as an “ultraclean rectifier”) in the foregoing gas purification facility, it is possible to obtain an electronic device manufacturing gas with a very small moisture content. By setting the moisture content in an electronic device manufacturing gas, particularly a plasma CVD gas, to 50 vol ppb or less, preferably 40 vol ppb or less, and particularly preferably 30 vol ppb or less, it is possible to prevent generation of a corrosive gas caused by moisture from a formed CVD film and a reduction in adhesion of the CVD film.

Generally, the airtightness of a rectifier depends on the machining accuracy of the rectifier and the materials and shapes of a rectifier body and a gasket, and its leak check requires a method that is suitable for its accuracy. This is because if the leak check accuracy is low, it is not possible to check whether or not bolts are evenly tightened when assembling a rectifier so as to prevent leakage from a pipe joint portion or a flange joining portion, and so on. Conventionally, it has been a general leak check method that, after assembling a rectifier, the inside of the rectifier is brought into a pressurized state with an inert gas such as nitrogen and then soapy water is applied to seams of a flange and so on, thereby observing generation of bubbles. However, with this method, a rectifier with particularly high airtightness (ultraclean rectifier) cannot be obtained and, even if rectification is repeatedly carried out, it is difficult to cause the moisture amount in a plasma CVD gas to be 1 vol ppm or less. In view of this, the present inventors have found that a rectifier with airtightness particularly higher than conventional (ultraclean rectifier) can be obtained and, as a result, the moisture amount in a plasma CVD gas can be made 50 vol ppb or less by the use of a rectifier leak check method wherein, after assembling a rectifier, a He leak detector being a mass detector exclusively for He is attached between the rectifier and an evacuator (vacuum pump) and then a He gas is sprayed onto a pipe joint portion or a flange joining portion, thereby detecting leakage at the pipe joint portion or the flange joining portion.

Hereinbelow, description will be made in more detail of an electronic device manufacturing gas, particularly a plasma CVD gas, of which the moisture content is very small, and a production method thereof.

FIG. 6 shows the gas purification facility 14 of the gas production facility shown in FIG. 1. The gas purification facility 14 shown in FIG. 6 is a SUS316L rectifier having been subjected to electrolytic polishing and comprises a column portion (Helipack-packed column) 141, a distillation pot 142, a reflux condenser 143, and a receiver 144. Normally, a feed gas composed of unsaturated fluorinated hydrocarbon is supplied to the Helipack-packed column 141. The distillation pot 142 is heated to a boiling point or higher of unsaturated fluorinated hydrocarbon. By feeding dry nitrogen to an upper portion of the reflux condenser 143, exhausting it to the outside of the system, and circulating cooling water to the reflux condenser 143, the feed gas with only a little moisture supplied from the Helipack-packed column 141 is cooled and condensed in the reflux condenser 143 and then is collected in the receiver 144 as a plasma CVD gas. The collected plasma CVD gas is filled into the gas supply container 18 (FIG. 1) by the gas filling facility 16. In terms of moisture removal performance, the dry nitrogen contains moisture of preferably 100 vol ppb or less, more preferably 10 vol ppb or less, and particularly preferably 1 vol ppb or less.

A He leak detector 145 being a mass detector exclusively for He is connected to the receiver 144 when performing a leak check of the gas purification facility 14 of FIG. 6. By spraying He onto a joint (in the example shown in FIG. 6, a joint between the Helipack-packed column 141 and the reflux condenser 143) and detecting He by the He leak detector 145 if there is leakage from the outside to the inside, the presence of leakage is confirmed.

What is most important for increasing the airtightness of the gas purification facility 14 is a flange joining portion forming the foregoing joint between the Helipack-packed column 141 and the reflux condenser 143. On the other hand, in order to avoid incorporation of impurity gases and particles into the CVD gas, a gasket for use at the flange joining portion is preferably made of metal such as stainless steel, aluminum, or copper. In order to ensure the sufficient airtightness by the use of the metal gasket, use is preferably made of a base material of a knife-edge ConFlat flange (ICF flange), a groove VG flange adapted for a metal hollow O-ring or a metal hollow O-ring with an elastic spring (Helicoflex), or the like. Further, since sealing is achieved by plastically deforming the gasket when attaching the flange, even tightening is very important and preferable.

As described above, in the leak check, the degree of leakage can be confirmed by attaching the He leak detector 145 between the gas rectification facility 14 and a non-illustrated evacuator (vacuum pump) and spraying the He gas onto the pipe joint portion or the flange joining portion while evacuating the inside of the system, thereby measuring the external leak rate (the leak rate from the outside to the inside). The external leak rate is 1.0×10⁻⁸ Pa·m³/sec or less and preferably 1.0×10⁻¹⁰ Pa·m³/sec or less. When the external leak rate exceeds 1.0×10⁻⁸ Pa·m³/sec, there is incorporation of a very little moisture from the outside so that the moisture content in the gas increases.

As described above, in this invention, for example, by the use of the gas purification facility 14 shown in FIG. 6, it is possible to obtain a plasma CVD gas composed of unsaturated fluorinated hydrocarbon and having a moisture content of 50 vol ppb or less.

The electronic device manufacturing gas, particularly the plasma CVD gas, of this invention contains an unsaturated fluorinated carbon compound of normally 90 wt % or more, preferably 95 wt % or more, more preferably 99 wt % or more, and particularly preferably 99.9 wt % or more. The plasma CVD gas of this invention may also contain another kind of plasma CVD gas or diluent gas within a range not impeding the object of this invention, but it is preferable not to contain a component other than the unsaturated fluorinated carbon compound.

As a method of obtaining an unsaturated fluorinated carbon compound containing a hydrogen atom-containing compound, in the case of octafluorocyclopentene as an example, as described in Unexamined Patent Publication No. Hei 9-95458, octafluorocyclopentene with a purity of 99.8 to 99.98% is obtained by reacting 1,2-dichlorohexafluorocyclopentene with potassium fluoride in dimethylholmamide in a nitrogen stream and extracting a product from a rectifier (conventional level airtightness) equipped in a reactor. The octafluorocyclopentene thus obtained is repeatedly subjected to precision distillation in a rectifier (conventional level airtightness) having a number of stages, thereby obtaining octafluorocyclopentene containing moisture of about 1 to 35 vol ppm.

In the case of octafluoro-2-pentyne as an example, as described in Unexamined Patent Publication No. 2003-146917 (EP Laid-Open Publication No. 1453082), octafluoro-2-pentyne with a purity of 99.9% or more containing moisture of about 1 to 60 vol ppm is obtained by contacting 2,3-dihydrodecafluoropentane and molten potassium hydroxide with each other, collecting a produced gaseous compound into a cooled trap, and then repeatedly subjecting the collected crude product to precision distillation in a rectifier (conventional level airtightness).

Although there is a case where the electronic device manufacturing gas, particularly the plasma CVD gas, of this invention contains a very little nitrogen gas and oxygen gas as gas components, the total amount of the nitrogen gas and oxygen gas is preferably 30 wt ppm or less by the plasma CVD gas weight standard.

The electronic device manufacturing gas, particularly the plasma CVD gas, of this invention is filled into an optional container so as to be offered for plasma reaction in the semiconductor manufacturing process or the like. When causing the plasma reaction, the plasma CVD gas of this invention is normally supplied along with an inert gas such as helium, neon, argon, or xenon in a plasma CVD apparatus. These inert gases each have a plasma CVD gas dilution effect and an effect of changing the electron temperature and electron density of a plasma and hence it becomes possible to control the balance between radicals and ions in the plasma reaction, thereby obtaining proper film forming conditions. The supply amount of the inert gas in the plasma CVD apparatus is normally 2 to 100 moles and preferably 5 to 20 moles relative to 1 mole of the plasma CVD gas of this invention.

The CVD using the plasma CVD gas of this invention represents activating the unsaturated fluorinated carbon compound by plasma discharge to produce active species such as ions and radicals, thereby forming a fluorocarbon polymer film on the surface of a processing object. Although the process of the formation of the polymer film is not entirely clear, it is considered that the generation of ion and radical species and various reactions such as polymerization and ring-opening reactions of the unsaturated fluorinated carbon compound are complexly related under the condition of electrolytic dissociation/dissociation. The object to be processed is not particularly limited, but is an article for use in the semiconductor manufacturing field, the electrical/electronic field, or the precision machine field, or, in terms of function, an article or the surface of a member that requires insulating properties, water repellency, corrosion resistance, acid resistance, lubricity, antireflection, or the like. Among them, it is particularly suitably used for forming an insulating film or an insulating material layer in the semiconductor device manufacturing process or forming a protective film of an organic electroluminescence element. As specific examples, there are cited formation of an interlayer insulating film on metal wiring of aluminum, copper, tungsten, or the like and a passivation film serving to protect an element, and so on. As the technique of plasma CVD, use can be made of a method described, for example, in Unexamined Patent Publication No. Hei 9-237783 or the like. As the plasma generating conditions, the conditions are normally adopted wherein the high frequency power applied to an upper electrode (shower head) of parallel flat plates is 10 W to 10 kW, the processing object temperature is 0 to 500° C., and the reaction chamber pressure is 0.0133 Pa to 13.3 kPa. The thickness of a deposited film is normally in the range of 0.01 to 10 μm. As the apparatus for use in plasma CVD, the parallel flat-plate type CVD apparatus is popular, but use can be made of a microwave CVD apparatus, an ECR-CVD apparatus, an inductive coupling plasma (ICP) CVD apparatus, or a high-density plasma CVD apparatus (helicon wave type, high frequency inductive type).

### EXAMPLE

Hereinbelow, this invention will be described in detail in terms of examples, but the contents of this invention are not limited thereto. Herein, the analysis conditions are common in the following examples and comparative examples, which are as follows. Further, analysis values in the following examples and comparative examples are each derived by rounding to the nearest whole number.

(Analysis 1) Conditions of Gas Chromatography Analysis (Hereinafter Abbreviated as “GC Analysis”)
Apparatus: HP6890 manufactured by Hewlett-Packard Company
Column: Ultra Alloy+−1 (s)
   (length 50 m, inner diameter 0.25 mm, film thickness 1.5 μm)
Column Temperature: fixed at −20° C. for 10 minutes and then raised to 200° C. in 30 minutes
Injection Temperature: 200° C.
Carrier Gas: Helium (flow rate 1 ml/min)
Detector: FID
Internal Standard: n-butane was used

(Analysis 2) Conditions of Karl Fischer Moisture Analysis (hereinafter abbreviated as “KF Analysis”)
Apparatus: AQ-7 manufactured by Hiranuma Sangyo Co., Ltd.
Generating Solution: Hydranal Aqualyte RS
Counter Electrode Solution: Aqualyte CN
Detection Limit: 0.5 wt ppm

(Analysis 3) Conditions of Gas Chromatography-Mass Spectrometry (Hereinafter Abbreviated as “GC-MS Analysis”)

<Gas Chromatography Portion>
Apparatus: HP-6890 manufactured by Hewlett-Packard Company
Column: Frontier Lab Ultra ALLOY⁺−1 (s)
   60 m×I.D 0.25 mm, 0.4 μmdf
Column Temperature: −20° C.
Carrier Gas: Helium

<Mass Spectrometer Portion>
Apparatus: 5973 NETWORK manufactured by Hewlett-Packard Company
Detector: EI Type (acceleration voltage: 70 eV)

(Analysis 4) Conditions of Highly-Sensitive Moisture Measuring Apparatus Cavity Ring-Down Spectroscopy (hereinafter abbreviated as “CRDS Analysis”)
Apparatus: MTO-1000H₂O manufactured by Tiger Optics
Detection Limit: 0.2 vol ppb

(Analysis 5) Conditions of Thermal Desorption Spectroscopy (Hereinafter Abbreviated as “TDS Analysis”)
Apparatus: WA 1000S manufactured by Denshi Kagaku Co., Ltd.
Heating Rate: 60° C./min

### EXAMPLE 1

In this Example 1, a ferritic stainless steel pipe (commercial product) having a Cr content of 29.1 wt % was electrolytically polished on its inner surface and used. The outer diameter of the pipe was ¼ inches, the length of the pipe was 1 m, and the surface roughness was 0.5 μm in terms of a center line average roughness Ra. After the electrolytic polishing, the foregoing stainless steel was charged into a furnace and the temperature was raised from room temperature to 550° C. in 1 hour while causing an Ar gas having an impurity concentration of several vol ppb or less to flow in the furnace, and then baking was carried out at that temperature for 1 hour to remove adhering moisture from the surface. After the baking was finished, the gas was switched to an oxidizing gas having a hydrogen concentration of 10% and a moisture concentration of 100 vol ppm and heat treatment was carried out for 3 hours. Part of the foregoing pipe was cut out and it was confirmed by XPS analysis that 100% Cr₂O₃ was formed on the inner surface of the pipe in a thickness of about 15 nm in the depth direction.

### EXAMPLE 2

In this Example 2, an austenitic stainless steel pipe (commercial product) having an Al content of 4.0 wt % was electrolytically polished on its inner surface and used. The pipe having the same size and the same surface roughness as those in Example 1 was used. After the electrolytic polishing, the foregoing stainless steel was charged into a furnace and the temperature was raised from room temperature to 400° C. in 1 hour while causing an Ar gas having an impurity concentration of several vol ppb or less to flow in the furnace, and then baking was carried out at that temperature for 1 hour to remove adhering moisture from the surface. After the baking was finished, the gas was switched to an oxidizing gas having a moisture concentration of 5 vol ppm and further added with 10 vol % of hydrogen in the moisture mixed gas and oxidation treatment was carried out at a treatment temperature of 900° C. for a treatment time of 1 hour. Part of the foregoing pipe was cut out and it was confirmed by XPS analysis that 100% Al₂O₃ was formed on the inner surface of the pipe in a thickness of about 200 nm in the depth direction.

### Comparative Example 1

The inner surface of a SUS316 pipe having the same size as that of the stainless steel pipe processed in Example 1 or 2 was annealed to obtain Ra=3 μm.

{Thermal Decomposition Characteristic Evaluation 1 of Fluorinated Hydrocarbon}

Using the stainless steel pipes obtained in Examples 1 and 2 (shown by “Cr₂O₃” and “Al₂O₃” in FIG. 4), a SUS-316L pipe of the same size whose inner surface was electrolytically polished (Ra=0.5 μm, shown by “SUS316L-EP” in FIG. 4), and the pipe of Comparative Example 1 (shown by “SUS316-BA” in FIG. 4), the thermal decomposition characteristics of a fluorinated carbon compound were evaluated. As the fluorinated carbon compound, use was made of octafluorocyclopentene (purity 99.95 vol %, moisture content 0.5 wt ppm or less). For the evaluation, use was made of an evaluation apparatus as shown in FIG. 3. At first, after connecting each pipe, to be evaluated, to the apparatus, impurities adsorbed to the inner surface of the pipe were removed by heating it at 500° C. for 1 hour while circulating an Ar gas having an impurity concentration of several ppb or less. After dropping the pipe temperature to room temperature, a test gas having a fluorinated carbon compound concentration adjusted to 1000 vol ppm was introduced into the evaluation apparatus at 5 cc/min by a gas flow rate controller. After the test gas was conducted to the pipe, FT-IR analysis was carried out to confirm that the test gas reached a detecting portion with the concentration of 1000 vol ppm. Thereafter, the pipe was heated from room temperature to 700° C. in 135 minutes. During the temperature rise, monitoring was constantly carried out by the use of a Fourier transform infrared spectrophotometer to measure the change in peak height caused by the fluorinated carbon compound. The results are shown in FIG. 4.

{Thermal Decomposition Characteristic Evaluation 2 of Fluorinated Hydrocarbon}

Evaluation was carried out in the same manner as Thermal Decomposition Characteristic Evaluation 1 except that octafluoro-2-pentyne (purity 99.99 vol %, moisture content 0.5 wt ppm or less) was used as a fluorinated carbon compound. The results are shown in FIG. 5.

From the evaluation results (FIGS. 4 and 5) of Thermal Decomposition Characteristic Evaluation 1 and 2 of Fluorinated Hydrocarbon, it has been found that, in the case of the stainless steel pipe having the electrolytically polished inner surface or the stainless steel pipe further subjected to the formation of the Cr₂O₃ or Al₂O₃ passivation surface, the decomposition start temperature of the fluorinated carbon compound is raised by about 50 to 200° C. as compared with the stainless steel pipe subjected to the normal annealing. Further, it has been found that the Al₂O₃ passivation surface largely raises the decomposition start temperature regardless of the kind of fluorinated carbon compound.

### EXAMPLE 3

After the inner surface of a bomb (commercial product) with a capacity of 1 liter made of a ferritic stainless steel having a Cr content of 29.1 wt % was electrochemically polished (Ra=0.5 μm), the foregoing bomb was charged into a furnace and the temperature was raised from room temperature to 550° C. in 1 hour while causing an Ar gas having an impurity concentration of several ppb or less to flow in the furnace, and then baking was carried out at that temperature for 1 hour to remove adhering moisture from the surface. After the baking was finished, the gas was switched to an oxidizing gas having a hydrogen concentration of 10 vol % and a moisture concentration of 100 vol ppm and heat treatment was carried out for 3 hours.

### EXAMPLE 4

After the inner surface of a bomb (commercial product) with a capacity of 1 liter made of an austenitic stainless steel having an Al content of 4.0 wt % was electrochemically polished (Ra=0.5 μm), the foregoing bomb was charged into a furnace and the temperature was raised from room temperature to 400° C. in 1 hour while causing an Ar gas having an impurity concentration of several vol ppb or less to flow in the furnace, and then baking was carried out at that temperature for 1 hour to remove adhering moisture from the surface. After the baking was finished, the gas was switched to an oxidizing gas having a moisture concentration of 5 vol ppm and further added with 10 vol % of hydrogen in the moisture mixed gas and oxidation treatment was carried out at a treatment temperature of 900° C. for a treatment time of 1 hour.

{Filling of High-Purity Fluorinated Carbon Compound}

After mounting a valve to each of the bombs of Examples 3 and 4, it was confirmed by an airtightness test that there was no gas leak. Highly purified octafluorocyclopentene (purity 99.93 vol %, moisture content 0.5 wt ppm or less) was filled into these bombs.

{Evaluation 1 of Bomb}

The filled gas was sampled from an outlet of the bomb valve and then cooled by the use of liquid nitrogen so as to be liquefied. The purity of the liquefied octafluorocyclopentene was measured by GC analysis. Further, the containing moisture amount was measured by KF analysis. This operation was carried out twice, i.e. immediately after the filling and after the lapse of 30 days from the filling. The results are shown in Table 1.

### Comparative Example 2

Instead of the bomb produced in Example 4, use was made of a stainless SUS316 steel bomb of the same size whose inner surface was annealed (Ra=3.5 μm). The results are shown in Table 1.

TABLE 1

| | | | | | | | | Moisture Amount | |
|---|---|---|---|---|---|---|---|---|---|
| | | | | Inner | | Purity (%) | | (wt ppm) | |
| | | Bomb | Inner | Surface | | | After | | After |
| | Bomb | Capacity | Surface | Passive | Filled Fluorinated | Immediately | 30 | Immediately | 30 |
| | Meterial | “L” | Treatment | Film | Carbon Compound | After Filling | Days | After Filling | Days |
| Example 3 | Ferritic | 1 | Electrochemical | Cr₂O₃ | octafluoro- | 99.93 | 99.93 | 0.5 or less | 0.5 or |
| | Stainless | | Polishing | | cyclopentene | | | | less |
| | Steel | | | | | | | | |
| Example 4 | Austenitic | 1 | Electrochemical | Al₂O₃ | | 99.93 | 99.93 | 0.5 or less | 0.5 or |
| | Stainless | | Polishing | | | | | | less |
| | Steel | | | | | | | | |
| Comparative | Austenitic | 1 | Annealing | Non | | 99.93 | 99.93 | 0.5 or less | 2.0 |
| Example 2 | Stainless | | | | | | | | |
| | Steel | | | | | | | | |

{Evaluation 2 of Bomb}

Using the bombs produced in Examples 3 and 4 and Comparative Example 2, evaluation was carried out in the same manner as Evaluation 1 of Bomb except that octafluoro-2-pentyne (purity 99.98 vol %, moisture content 0.5 wt ppm or less) was used as a high-purity fluorinated carbon compound to be filled. The results are shown in Table 2.

TABLE 2

| | | | | | | | | Moisture Amount | |
|---|---|---|---|---|---|---|---|---|---|
| | | | | Inner | | Purity (%) | | (wt ppm) | |
| | | Bomb | Inner | Surface | | | After | | After |
| | Bomb | Capacity | Surface | Passive | Filled Fluorinated | Immediately | 30 | Immediately | 30 |
| | Meterial | “L” | Treatment | Film | Carbon Compound | After Filling | Days | After Filling | Days |
| Example 3 | Ferritic | 1 | Electrochemical | Cr₂O₃ | otafluoro- | 99.98 | 99.98 | 0.5 or less | 0.6 |
| | Stainless | | Polishing | | 2-Pentyne | | | | |
| | Steel | | | | | | | | |
| Example 4 | Austenitic | 1 | Electrochemical | Al₂O₃ | | 99.98 | 99.98 | 0.5 or less | 0.5 or |
| | Stainless | | Polishing | | | | | | less |
| | Steel | | | | | | | | |
| Comparative | Austenitic | 1 | Annealing | Non | | 99.98 | 99.93 | 0.5 or less | 4.5 |
| Example 2 | Stainless | | | | | | | | |
| | Steel | | | | | | | | |

From the results of Tables 1 and 2, there was observed no reduction in purity or no increase in moisture content with respect to the fluorinated carbon compound filled in the bomb whose inner surface roughness was set to Ra=0.5 μm and whose inner surface was subjected to the passivation with Cr₂O₃ or Al₂O₃.

### EXAMPLE 5

Octafluorocyclopentene having a purity of 99.95 vol % and a moisture content of 35 vol ppm was prepared as a raw material and use was made, as an ultraclean rectifier, of the electrolytically polished SUS316L rectification facility 14 having the Helipack-packed column 141 of 80 stages as the theoretical number of stages (in FIG. 6, the inner surface roughness of the column portion 141, the rectification pot portion 142, the reflux condensing portion 143, and so on was set to Ra=0.5 μm or less, the Helipack was set to Ra=1.0 μm by chemical polishing, and the external leak rate was set to 1.0×10⁻¹⁰ Pa·m³/sec or less).

34.5 parts of the foregoing octafluorocyclopentene were charged into the ultraclean rectifier. Cooling water of 0° C. was circulated to the reflux condensing portion 143, the rectification pot was heated by a heating medium of 32° C., and dry nitrogen (moisture amount 1 vol ppb or less) was fed to the upper portion of the reflux condenser 143 at a flow rate of 50 cc/min and discharged to the outside of the system. The total reflux was carried out at normal pressure for 1 hour. Thereafter, a fraction was extracted at a reflux ratio of 40:1 and 18.5 parts of octafluorocyclopentene were collected in the receiver 144. The moisture value by CRDS analysis was 18 vol ppb.

### EXAMPLE 6

An experiment was carried out in the same manner as in Example 5 except that octafluoro-2-pentyne (purity 99.99 vol %, moisture content 60 vol ppm) was used as a raw material and the inner pressure was set to 0.15 MPa in terms of absolute pressure, thereby collecting 20.7 parts of octafluoro-2-pentyne. The moisture value by CRDS analysis was 25 vol ppb.

### EXAMPLE 7

Using a silicon oxide film wafer partly deposited with aluminum as a substrate, using a parallel flat-plate type plasma CVD apparatus as a plasma CVD apparatus, and using the plasma CVD gas produced in Example 5, plasma CVD of an insulating film was carried out under the following conditions.

Plasma CVD Gas Flow Rate: 40 sccm
Argon Flow Rate 400 sccm, Pressure: 250 mTorr
RF Output (Frequency 13.56 MHz): 400 W
Substrate Temperature 250° C.

A film (fluorocarbon film) having a thickness of 0.5 μm was obtained on the substrate processed under the foregoing conditions. This film (fluorocarbon film) was not subjected to occurrence of voids, was fine and uniform, and was excellent in adhesion to the substrate. The relative permittivity of the film was 2.2. The results of TDS analysis are shown in FIG. 7.

### Comparative Example 3

An experiment was carried out in the same manner as in Example 7 except that octafluorocyclopentene (purity 99.95 vol %, moisture content 35 vol ppm, corresponding to the raw material supplied to the ultraclean rectifier in Example 5) was used as a plasma CVD gas, thereby obtaining a film having a thickness of 0.5 μm on a substrate. This film was not subjected to occurrence of voids and was fine and uniform, but the relative permittivity of the film was 2.4. The results of TDS analysis are shown in FIG. 7.

Referring to FIG. 7, in Comparative Example 3, at a substrate temperature of 200° C. or more, a gas is released from the film on the substrate so that the pressure increases, while, in Example 7, even at a substrate temperature of 200° C. or more, a gas is not released so much from the film on the substrate that the pressure does not increase. Since the film on the substrate obtained in Example 6 contains less gas, it is possible to prevent film stripping or metal corrosion caused by generation of hydrogen fluoride.

### EXAMPLE 8

An experiment was carried out in the same manner as in Example 7 except that the gas produced in Example 6 was used as a plasma CVD gas, thereby obtaining a film having a thickness of 0.5 μm on a substrate. This film was not subjected to occurrence of voids, was fine and uniform, and was excellent in adhesion to the substrate. The relative permittivity of the film was 2.2.

### INDUSTRIAL APPLICABILITY

This invention is applicable to production facilities adapted to produce various feed gases for use in the manufacture of electronic devices such as semiconductor devices and liquid crystal display devices and to supply containers thereof, thereby reducing impurities incorporated into the feed gases.

1. A gas production facility, wherein a surface roughness of a portion of the gas production facility that contacts with a gas for manufacturing an electronic device is 1 μm or less in terms of a center line average roughness Ra.

2. The gas production facility according to claim 1, wherein an oxide passivation film of at least one selected from the group consisting of aluminum oxide, chromium oxide, titanium oxide, yttrium oxide, and magnesium oxide, is formed on an inner surface of said gas production facility.

3. The gas production facility according to claim 1, wherein an inner surface of said gas production facility has an oxide passivation film formed by contacting with an oxidizing gas and carrying out heat treatment.

4. The gas production facility according to claim 1, wherein an inner surface of said gas production facility has an oxide passivation film formed by carrying out a thermal spraying process.

5. The gas production facility according to claim 1, wherein the gas for manufacturing the electronic device comprises a fluorinated carbon compound of which a ratio (F/C ratio) between the number of fluorine atoms and the number of carbon atoms is 1.0 to 2.0.

6. A method of producing a fluorinated carbon compound, comprising the step of using the gas production facility as defined in claim 1.

7. The method of producing a fluorinated carbon compound according to claim 6, wherein said fluorinated carbon compound is at least one selected from the group consisting of tetrafluoroethylene, hexafluoropropene, tetrafluoropropyne, hexafluorocyclobutene, hexafluoro-1,3-butadiene, hexafluoro-1-butyne, hexafluoro-2-butyne, octafluorocyclobutane, octafluorocyclopentene, octafluoro-1,3-pentadiene, octafluoro-1,4-pentadiene, octafluoro-1-pentyne, octafluoro-2-pentyne, and hexafluorobenzene.

8. A gas supply container wherein a surface roughness of a portion of the gas supply container that contacts with a gas for manufacturing an electronic device is 1 μm or less in terms of a center line average roughness Ra.

9. The gas supply container according to claim 8, wherein an oxide passivation film of at least one selected from the group consisting of aluminum oxide, chromium oxide, titanium oxide, yttrium oxide, and magnesium oxide is formed on an inner surface of said gas supply container.

10. The gas supply container according to claim 8, wherein an inner surface of said gas supply container has an oxide passivation film formed by contacting with an oxidizing gas and carrying out heat treatment.

11. The gas supply container according to claim 8, wherein an inner surface of said gas supply container has an oxide passivation film formed by carrying out a thermal spraying process.

12. The gas supply container according to claim 8, wherein the gas for manufacturing the electronic device comprises a fluorinated carbon compound.

13. A method of supplying a fluorinated carbon compound, comprising the step of using the gas supply container as defined in claim 8.

14. The method of supplying a fluorinated carbon compound according to claim 13, wherein said fluorinated carbon compound is one selected from the group consisting of tetrafluoroethylene, hexafluoropropene, tetrafluoropropyne, hexafluorocyclobutene, hexafluoro-1,3-butadiene, hexafluoro-1-butyne, hexafluoro-2-butyne, octafluorocyclobutane, octafluorocyclopentene, octafluoro-1,3-pentadiene, octafluoro-1,4-pentadiene, octafluoro-1-pentyne, octafluoro-2-pentyne, and hexafluorobenzene.

15. A gas for manufacturing an electronic device comprising an unsaturated fluorinated hydrocarbon having a moisture content of 50 vol ppb or less.

16. The gas for manufacturing the electronic device according to claim 15, wherein said gas for manufacturing the electronic device is a plasma CVD gas.

17. The plasma CVD gas according to claim 16, wherein said unsaturated fluorinated hydrocarbon is at least one selected from the group consisting of octafluorocyclopentene, octafluoro-2-pentyne, octafluoro-1,4-pentadiene, and hexafluoro-1,3-butadiene.

18. A production method of a gas for manufacturing an electronic device, comprising the step of carrying out distillation using a rectifier with an external leak rate of 1.0×10⁻⁸ Pa·m³/sec or less in the gas production facility as defined in claim 2.

19. The production method according to claim 18, wherein said gas for manufacturing an electronic device is a plasma CVD gas.

20. A fluorocarbon film manufacturing method comprising the step of using the gas for manufacturing the electronic device defined in claim 16.
